(19) United States
(12) Patent Application Publication
(10) Pub. No.: US 2024/0164874 A1
(43) Pub. Date: May 23, 2024

(54) MODELING DENTAL STRUCTURES FROM DENTAL SCAN

(71) Applicant: GET-GRIN INC., Airmont, NY (US)

(72) Inventors: Alon Luis LIPNIK, Tel Aviv (IL); Yarden EILAT-BLOCH, Haifa (IL); Oded KRAMS, Tel Aviv (IL); Adam Benjamin SCHULHOF, New City, NY (US); Carmi RAZ, Gizo (IL)

(21) Appl. No.: 18/424,169

(22) Filed: Jan. 26, 2024

Related U.S. Application Data
(63) Continuation of application No. PCT/US22/38943, filed on Jul. 29, 2022.
(60) Provisional application No. 63/227,066, filed on Jul. 29, 2021.
(60) Provisional application No. 63/358,544, filed on Jul. 6, 2022.

(51) Int. Cl. A61C 9/00; G06T 7/00; G16H 30/40

(52) U.S. Cl. CPC A61C 9/0053; G06T 7/00; G16H 30/40; G06T 2207/20081; G06T 2207/30036

(57) ABSTRACT

A method for updating a three-dimensional (3D) dental model of at least one tooth, comprising: (a) providing at least one 2D dental image including the at least one tooth; (b) running a trained visual filter neural network on the 2D dental image to identify the tooth number of the at least one tooth; (c) providing a baseline 3D dental model that includes the at least one tooth; (d) generating a 2D capture of the baseline 3D dental model; (e) updating the 2D capture of the 3D dental model to include the identified tooth number obtained from the 2D dental image; and (f) using the updated 2D capture to update the 3D dental to include the identified tooth number obtained from the 2D dental image.

### CROSS-REFERENCE TO RELATED APPLICATIONS

The present application is a continuation of PCT Application No. PCT/US2022/038943 filed Jul. 29, 2022, which claims the benefit of U.S. Provisional Application Ser. No. 63/227,066 filed Jul. 29, 2021, and U.S. Provisional Application Ser. No. 63/358,544 filed Jul. 6, 2022, each of which is incorporated by reference herein in its entirety.

### TECHNICAL FIELD

The systems and methods described herein relate to dental structure modeling, and more specifically a method and system for modeling a dental structure from a video dental scan.

### BACKGROUND

Dental professionals and orthodontists may treat and monitor a patient's dental condition based on in-person visits. Treatment and monitoring of a patient's dental condition may require a patient to schedule multiple in-person visits to a dentist or orthodontist. The quality of treatment and the accuracy of monitoring may vary depending on how often and how consistently a patient sees a dentist or orthodontist. In some cases, suboptimal treatment outcomes may result if a patient is unable or unwilling to schedule regular visits to a dentist or orthodontist.

### SUMMARY

Recognized herein is a need for remote dental monitoring solutions to allow dental patients to receive high quality dental care, without requiring a dental professional to be physically present with the patient. Some dental professionals and orthodontists may use conventional teledentistry solutions to accommodate patients' needs and schedules. However, such conventional teledentistry solutions may provide inadequate levels of supervision. Further, such conventional teledentistry solutions may be limited by an inaccurate or insufficient monitoring of a patient's dental condition based on one or more photos taken by the patient, if the photos do not adequately capture various intraoral features.

The present disclosure provides methods and systems that are capable of generating (or configured to generate) a three-dimensional (3D) model of a dental structure of a dental patient from a video of a dental scan collected using a mobile device. The 3D model may be a 3D surface model (mesh) with fine details of the surface of the dental structure. The 3D model reconstructed from the videos as described herein can have substantially the same or similar quality and surface details as those of a 3D model (e.g., optical impressions) produced using an existing high-resolution clinical intraoral scanner. It is noted that high-resolution clinical intraoral scans can be time-consuming and uncomfortable to the patient.

Methods and systems of the present disclosure beneficially provide a convenient and efficient solution for monitoring and evaluating the positions of a patient's teeth during the course of orthodontic treatment using a user mobile device, in the comfort of the patient's home or another convenient location, without requiring the patient to travel to a dental clinic or undergo a time-consuming and uncomfortable full clinical intraoral dental scan.

In an aspect, provided herein is a method for training a visual filter neural network to identify one or more tooth numbers of one or more teeth from one or more dental images, comprising: (a) providing an intraoral region model, wherein the intraoral region model comprises one or more model teeth; (b) providing orientation data, wherein the orientation data correlates a spatial location of the one or more model teeth with the corresponding tooth number of the one or more model teeth; (c) providing a plurality of training dental images, wherein each training dental image of the plurality of training dental images comprises one or more teeth; (d) creating a plurality of training datasets by using the visual information corresponding to the one or more model teeth to label the one or more teeth in each one of the plurality of training dental images with a respective label, wherein the respective label indicates either a tooth number or a tooth number is not identifiable; and (e) training the visual filter neural network based on the plurality of training datasets to identify a tooth within a dental image of a subject and label the tooth with a corresponding tooth number.

In some cases, the intraoral region model is a two-dimensional (2D) model representation of the intraoral region of an adult subject from a front perspective. In some cases, the intraoral region model is a 2D model representation of the intraoral region of an adult subject from a top view perspective. In some cases, the intraoral region model is a three-dimensional (3D) model representation of the intraoral region of an adult subject. In some cases, the intraoral region model is a 2D model representation of the intraoral region of a child subject from a front perspective. In some cases, the oral region model is a 2D model representation of the intraoral region of a child from a top view perspective. In some cases, the oral region model is a 3D model representation of the intraoral region of a child subject. In some cases, the orientation data is acquired from capturing the intraoral region model with a dental scope, and wherein the orientation data corresponds to the spatial orientation of the dental scope relative to the intraoral region being captured.

In some cases, the dental image is of a human subject. In some cases, the dental image is captured within the visible light spectrum. In some cases, the dental image is acquired using a dental scope.

In some cases, the creating of the plurality of training datasets comprises comparing and matching a rotation or orientation of a tooth in a training dental image with a rotation or orientation of the corresponding model tooth. In some cases, the creating of the plurality of training datasets comprises comparing and matching a scale of a tooth in a training dental image with a scale of the corresponding model tooth. In some cases, the creating of the plurality of training datasets comprises comparing and matching a contour of a tooth in a training dental image with a contour of the corresponding model tooth, wherein a contour of the tooth is determined from outlier pixel intensity values. In some cases, the creating of the plurality of training datasets comprises comparing and matching a color of a tooth in a training dental image with a color of the corresponding model tooth, wherein a color of the tooth is determined from pixel intensity values. In some cases, the creating of the plurality of training datasets comprises comparing and matching morphologic structure of a tooth in a training dental image with a morphologic structure of the corresponding model tooth, wherein the morphologic structure of the tooth is determined from the shape of the teeth and surface pixel color and intensity.

In some cases, the creating of the plurality of training datasets comprises identifying a first tooth in the training dental image based on the relation of the first tooth to a second tooth adjacent to the first tooth. In some cases, the creating of the plurality of training datasets comprises identifying a first tooth in the training dental image based on the relation of the first tooth to a second tooth opposite of the first tooth. In some cases, the method further comprises reviewing the respective label of a training dental image of the plurality of training dental images to confirm the accuracy of the label. In an aspect, provided herein is a method to identify a number of a tooth from a dental image, comprising: providing a dental image, wherein the dental image comprises a visible part of the tooth; and running a visual filter neural network to identify the tooth number. In some cases, the visual filter neural network is provided with an intraoral region model of a user, and wherein the dental image is of the user. In some cases, the dental image is projected on the identified tooth on the intraoral region model of the user.

In an aspect, provided herein is a method for updating a three-dimensional (3D) dental model of at least one tooth, comprising: (a) providing at least one two-dimensional (2D) dental image including the at least one tooth; (b) running a visual filter neural network on the 2D dental image to identify the tooth number of the at least one tooth; (c) providing a baseline 3D dental model that includes the at least one identified tooth; (d) generating a 2D capture of the baseline 3D dental model; (e) updating the 2D capture of the 3D dental model in accordance with the 2D dental image; and (f) using the updated 2D capture to update the 3D dental model.

In an aspect, provided herein is a method for updating an initial three-dimensional (3D) dental model of a dental structure of a subject, the method comprising: (a) providing a dental video scan of the dental structure of the subject captured using a camera of a mobile device, wherein the dental structure of the subject comprises one or more oral landmarks; (b) analyzing the dental video scan to identify an oral landmark of the one or more oral landmarks; (c) providing the initial 3D dental model of the dental structure of the subject; (d) comparing the dental scan video with the initial 3D dental model to determine differences between the identified oral landmark in the two models; and (e) updating the initial 3D dental model to include the differences of the identified oral landmark.

In some cases, the analyzing of the dental video scan comprises running a visual filter neural network to identify the tooth number of at least one tooth in the dental structure of the subject. In some cases, the analyzing of the dental video scan comprises determining the relative distance between a camera used to capture the dental video scan and the oral landmark identified in the dental video scan. In some cases, the identified oral landmark is the arch plane of a subject, and the relative distance comprises the distance from the arch plane to the camera used to capture the dental video scan. In some cases, the analyzing of dental video scan comprises determining the object distance and time duration of at least two perspectives within the dental video scan. In some cases, the analyzing of the dental video scan comprises identifying at least one focus object in a frame of the dental video scan, generating a perspective focus plane of the at least one focus object, and identifying the relative distance from the focus plane to the camera used to capture the dental video scan.

In some cases, the updating comprises: (i) applying structure from motion (SfM) to the dental video scan; (ii) applying a multi view stereo (MVS) algorithm of at least two perspectives to the dental video scan, (iii) determining a transformation of at least one element of the dental structure and applying the transformation to update a position of the at least one element in the 3D dental model; or (iv) deforming a surface of a local area of the at least one element of the dental structure using a deformation algorithm. In some cases, the 3D dental model is a generic model. In some cases, the 3D dental model comprises the dental structure of the subject. In some cases, the relative distance is retrieved from the dental video scan metadata.

In some cases, an aspect provided here in a non-transitory computer-readable medium comprising machine-executable instructions that, upon execution by one or more computer processors, implements a method for delivering context based information to a mobile device in real time, the method comprising: a memory for storing a set of instructions; and one or more processors configured to execute the set of instructions to: (a) provide a dental video scan of the dental structure of the subject using a camera of a mobile device, wherein the dental structure of the subject comprises one or more oral landmarks; (b) analyze the dental video scan to identify an oral landmark of the one or more oral landmarks; (c) provide the 3D dental model of the dental structure of the subject; (d) compare the dental scan video with the 3D dental model to determine differences between the identified oral landmark in the two models; and (e) update the 3D dental model to include the differences of the identified oral landmark.

In some cases, the analyzing of the dental video scan comprises running a visual filter neural network to identify the tooth number of at least one tooth in the dental structure of the subject. In some cases, the analyzing of the dental video scan comprises determining the relative distance between a camera used to capture the dental video scan and the oral landmark identified in the dental video scan. In some cases, the identified oral landmark is the arch plane of a subject, and the relative distance comprises the distance from the arch plane to the camera used to capture the dental video scan. In some cases, the analyzing of dental video scan comprises determining the object distance and time duration of at least two perspectives within the dental video scan. In some cases, the analyzing of the dental video scan comprises identifying at least one focus object in a frame of the dental video scan, generating a perspective focus plane of the at least one focus object, and identifying the relative distance from the focus plane to the camera used to capture the dental video scan.

In some cases, the updating comprises: (i) applying structure from motion (SfM) to the dental video scan; (ii) applying a multi view stereo (MVS) algorithm of at least two perspectives to the dental video scan, (iii) determining a transformation of at least one element of the dental structure and applying the transformation to update a position of the at least one element in the 3D dental model; or (iv) deforming a surface of a local area of the at least one element of the dental structure using a deformation algorithm. In some cases, the 3D dental model is a generic model. In some cases, the 3D dental model comprises the dental structure of the subject. In some cases, the relative distance is retrieved from the dental video scan metadata.

As used herein, the term “dental video scan” or “dental scan” refers to a video or an image frame from a video capture of the intraoral perspective of the teeth arch or of a tooth.

As used herein, the term “arch plane” refers to at least one imaginary plane that is generated form cut line crossing at least one mouth dental arch, or at the top of the teeth (up or bottom).

As used herein, the term “perspective focus plane” refers to at least one plane the generated by perspective of one camera shot or frame that capture image and the collection of objects that in the current focus of the camera. The “perspective focus plane” is an imaginary plane generated by the objects that are in the same focal distance from the camera in selected time.

The term “dental structure” as utilized here may include intra-oral structures or dentition, such as human dentition, individual teeth, quadrants, full arches, upper and lower dental arches (which may be positioned and/or oriented in various occlusal relationships relative to each other), soft tissue (e.g., gingival and mucosal surfaces of the mouth, or perioral structures such as the lips, nose, cheeks, and chin), bones, and any other supporting or surrounding structures proximal to one or more dental structures. Intra-oral structures may include both natural structures within a mouth and artificial structures such as dental objects (e.g., prosthesis, implant, appliance, restoration, restorative component, or abutment). Although the present methods and systems are described with respect to dentition and dental structures, it should be noted that the 3D model construction algorithms and methods described herein can be applied to various other applications where 3D modeling is desired.

Unless otherwise defined, all technical and scientific terms used herein have the same meaning as commonly understood by one of ordinary skill in the art to which the methods and systems described herein belongs. Although suitable methods and materials are described below, methods and materials similar or equivalent to those described herein can be used in the practice of the methods and systems described herein. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the methods and systems of the described herein may involve performing or completing selected tasks or steps manually, automatically, or a combination thereof. Moreover, according to actual instrumentation and equipment of preferred embodiments of methods, apparatus and systems described herein, several selected steps could be implemented by hardware or by software on any operating system of any firmware or a combination thereof. For example, as hardware, selected steps could be implemented as a chip or a circuit. As software, selected steps could be implemented as a plurality of software instructions being executed by a computer using any suitable operating system. In any case, selected steps of the methods and systems described herein could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the systems and methods described herein and see how they may be carried out in practice, embodiments will now be described, by way of non-limiting examples only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 schematically illustrates an example of a method for training a visual filter neural network, in accordance with some embodiments.

FIG. 2 schematically illustrates an example of a system to designate tooth number to a tooth on dental images, in accordance with some embodiments.

FIG. 3, schematically illustrates an example of a method for updating a three-dimensional (3D) point cloud of at least one tooth, in accordance with some embodiments.

FIG. 4 schematically illustrates a computer system that is programmed or otherwise configured to implement t at least some of the methods or the systems disclosed herein, in accordance with some embodiments.

### DETAILED DESCRIPTION

While various embodiments have been shown and described herein, it will be obvious to those skilled in the art that such embodiments are provided by way of example only. Numerous variations, changes, and substitutions may occur to those skilled in the art without departing from the methods and systems described herein. It should be understood that various alternatives to the embodiments described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

As utilized herein, terms “component,” “system,” “interface,” “unit” and the like are intended to refer to a computer-related entity, hardware, software (e.g., in execution), and/or firmware. For example, a component can be a processor, a process running on a processor, an object, an executable, a program, a storage device, and/or a computer. By way of illustration, an application running on a server and the server can be a component. One or more components can reside within a process, and a component can be localized on one computer and/or distributed between two or more computers.

As used herein, the term “visual filter neural network” corresponds to a neural network used to identify a number of a tooth from one or more dental images. In some cases, the visual filter neural network works by: (a) providing an intraoral region model, wherein the intraoral region model comprises one or more model teeth; (b) providing orientation data, wherein the orientation data correlates the spatial location of the one or more model teeth with the corresponding tooth number of the one or more model teeth; (c) associating the tooth number of the one or more model teeth with visual information corresponding to the one or more model teeth; (d) providing a plurality of training dental images, wherein each training dental image of the plurality of training dental images comprises one or more teeth; (e) using the visual information corresponding to the one or more model teeth to create a plurality of training datasets by labeling the one or more teeth in each one of the plurality of training dental images with a respective label indicating a tooth number or that a tooth number could not be identified; and (f) training the visual filter neural network based on the plurality of training datasets to identify a tooth within a dental image and label the tooth with a corresponding tooth number.

Further, these components can execute from various computer readable media having various data structures stored thereon. The components can communicate via local and/or remote processes such as in accordance with a signal having one or more data packets (e.g., data from one component interacting with another component in a local system, distributed system, and/or across a network, e.g., the Internet, a local area network, a wide area network, etc. with other systems via the signal).

### Overview

The present disclosure deals in various aspects of three dimensional (3D) digital representations of an individual's intraoral structure. Dental scans can be used to update such 3D representations of an individual's intraoral structure. In some cases, a visual filter neural network can be used to update the 3D representations.

### Visual Filter Neural Network

In an aspect, provided herein is a method for training a visual filter neural network to identify one or more tooth numbers of one or more teeth from one or more dental images, comprising: (a) providing an intraoral region model, wherein the intraoral region model comprises one or more model teeth; providing orientation data, wherein the orientation data correlates the spatial location of the one or more model teeth with the corresponding tooth number of the one or more model teeth; associating the tooth number of the one or more model teeth with visual information corresponding to the one or more model teeth; providing a plurality of training dental images, wherein each training dental image of the plurality of training dental images comprises one or more teeth; creating a plurality of training datasets by using the visual information corresponding to the one or more model teeth to label the one or more teeth in each one of the plurality of training dental images with a respective label, wherein the respective label indicates either a tooth number or a tooth number is not identifiable; and training the visual filter neural network based on the plurality of training datasets to identify a tooth within a dental image of a subject and label the tooth with a corresponding tooth number.

In some cases, the intraoral region model is a two-dimensional (2D) model representation of the intraoral region of an adult subject from a front perspective. In some cases, the intraoral region model is a 2D model representation of of the intraoral region of an adult subject from a top view perspective. In some cases, the intraoral region model is a three-dimensional (3D) model representation of the intraoral region of an adult subject. In some cases, the intraoral region model is a 2D model representation of of the intraoral region of a child subject from a front perspective. In some cases, the oral region model is a 2D model representation of the intraoral region of a child from a top view perspective. In some cases, the oral region model is a 3D model representation of the intraoral region of a child subject.

In some cases, the orientation data is acquired from capturing the intraoral region model with a dental scope, and wherein the orientation data corresponds to the spatial orientation of the dental scope relative to the intraoral region being captured. In some cases, the dental image is of a human subject. In some cases, the dental image is captured within the visible light spectrum. In some cases, the dental image is acquired using a dental scope.

In some cases, the creating of the plurality of training datasets comprises comparing and matching a rotation or orientation of a tooth in a training dental image with a rotation or orientation of the corresponding model tooth. In some cases, the creating of the plurality of training datasets comprises comparing and matching a scale of a tooth in a training dental image with a scale of the corresponding model tooth. In some cases, the creating of the plurality of training datasets comprises comparing and matching a contour of a tooth in a training dental image with a contour of the corresponding model tooth, wherein a contour of the tooth is determined from outlier pixel intensity values. In some cases, the creating of the plurality of training datasets comprises comparing and matching a color of a tooth in a training dental image with a color of the corresponding model tooth, wherein a color of the tooth is determined from pixel intensity values.

In some cases, the creating of the plurality of training datasets comprises identifying a first tooth in the training dental image based on the relation of the first tooth to a second tooth adjacent to the first tooth. In some cases, the creating of the plurality of training datasets comprises identifying a first tooth in the training dental image based on the relation of the first tooth to a second tooth opposite of the first tooth. In some cases, the method further comprises: reviewing the respective label of a training dental image of the plurality of training dental images to confirm the accuracy of the label.

In an aspect, the present disclosure provides a system for training a visual filter neural network for segmentation of type and number of a teeth from dental images comprising: providing an oral region model and a target orientation of a dental images defined by the classification neural network;
creating a training dataset by labeling each one of a plurality of dental images provided from a storage server with a respective label indicating teeth number or with a respective label indicating a tooth is not identified;
creating a second training dataset by labeling each one of a plurality of dental images provided from a storage server with a respective label indicating teeth number or with a respective label indicating a tooth is not identified;
providing additional dental images stored on the storage server;
training the visual filter neural network based on the training datasets for classifying the additional dental images into classification category indicating teeth number;
compare classified dental images on respective labels indicating teeth number or non-indicate and updating training sets.

In some embodiments the oral region model is a two-dimensional (2D) model representation of an adult teeth in front perspective. In some embodiments the oral region model is a 2D model representation of an adult teeth in up-view perspective.

In some embodiments the oral region model is a three-dimensional (3D) model representation of an adult teeth.

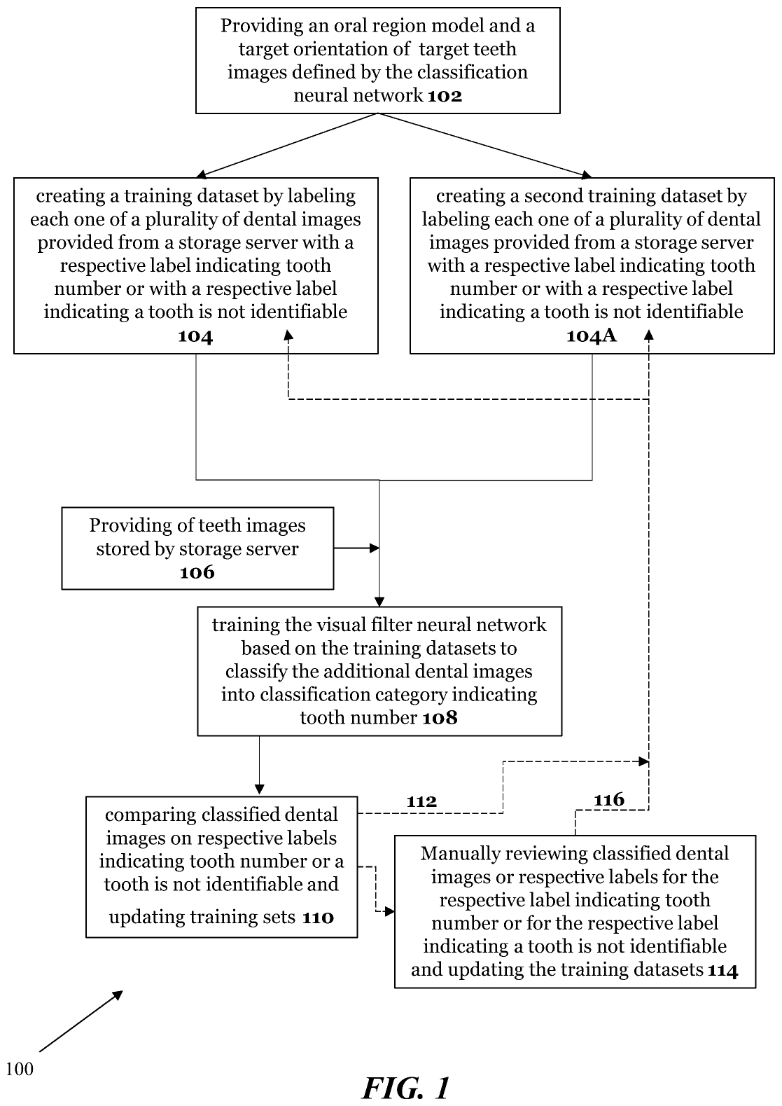

FIG. 1 schematically illustrates one example of a method for training a visual filter neural network 100 to classify the identify a tooth number from dental images. The method may include providing an oral region model and a target orientations of a dental images defined by the classification neural network 102; creating a training dataset by labeling each one of a plurality of dental images provided from a storage server with a respective label indicating teeth number or with a respective label indicating a tooth is not identifiable 104;
creating a second training dataset by labeling each one of a plurality of dental images provided from a storage server with a respective label indicating teeth number or with a respective label indicating non indicate 104A;
providing additional dental images stored on the storage server 106;
training the visual filter neural network based on the training datasets to classify the additional dental images into classification category indicating teeth number 108;
compere classified dental images on respective labels indicating teeth number or a tooth is not identifiable 110 and updating training sets 114. In some embodiments the method can further comprises reviewing classified dental images on respective labels for the respective label indicating teeth number or for the respective label indicating a tooth is not identifiable 114 and updating the training datasets 116. In some cases, reviewing is performed manually.

### Assigning Tooth Numbers on Dental Images

In an aspect provided herein is a method to identify a number of a tooth from a dental image, comprising: providing a dental image, wherein the dental image comprises a visible part of the tooth; and running a trained visual filter neural network to identify the tooth number. In some cases, the visual filter neural network is provided with an intraoral region model of a user, and wherein the dental image is of the user. In some cases, the dental image is projected on the identified tooth on the intraoral region model of the user.

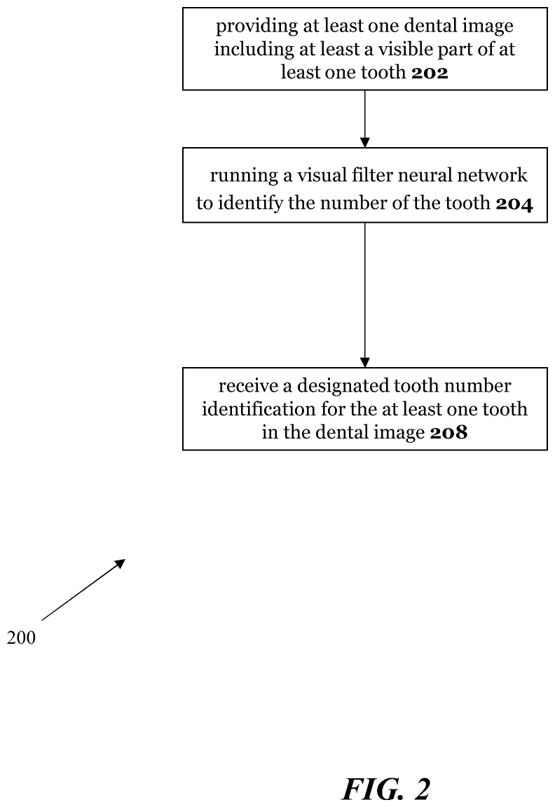

FIG. 2 schematically illustrate an example of a method 200 to designate teeth number to identify a number of a tooth from a dental image. In some cases, the method comprises providing at least one dental image including at least visible part of a at least one tooth 202; running a visual filter neural network to identify the tooth number 204; and receive a designated teeth number identification for the at least one tooth in the dental image 208.

### Updating a Three-Dimensional (3D) Dental Model

In an another aspect, provided herein is a method for updating a three-dimensional (3D) dental model of at least one tooth, comprising: (a) providing at least one two-dimensional (2D) dental image including the at least one tooth; (b) running a trained visual filter neural network on the 2D dental image to identify the tooth number of the at least one tooth; (c) providing a baseline 3D dental model that includes the at least one tooth; (d) generating a 2D capture of the baseline 3D dental model; (e) updating the 2D capture of the 3D dental model to include the identified tooth number obtained from the 2D dental image; and (f) using the updated 2D capture to update the 3D dental to include the identified tooth number obtained from the 2D dental image.

The present disclosure provides methods and systems that are capable of generating (or configured to generate) a three-dimensional (3D) model of a dental structure of a dental patient using video of dental scan collected using a mobile device. The 3D model may be a 3D surface model (mesh) with fine details of the surface of the dental structure. The 3D model reconstructed from the videos as described herein can have substantially the same or similar quality and surface details as those of a 3D model (e.g., optical impressions) produced using an existing high-resolution clinical intraoral scanner. It is noted that high-resolution clinical intraoral scans can be time-consuming and uncomfortable to the patient.

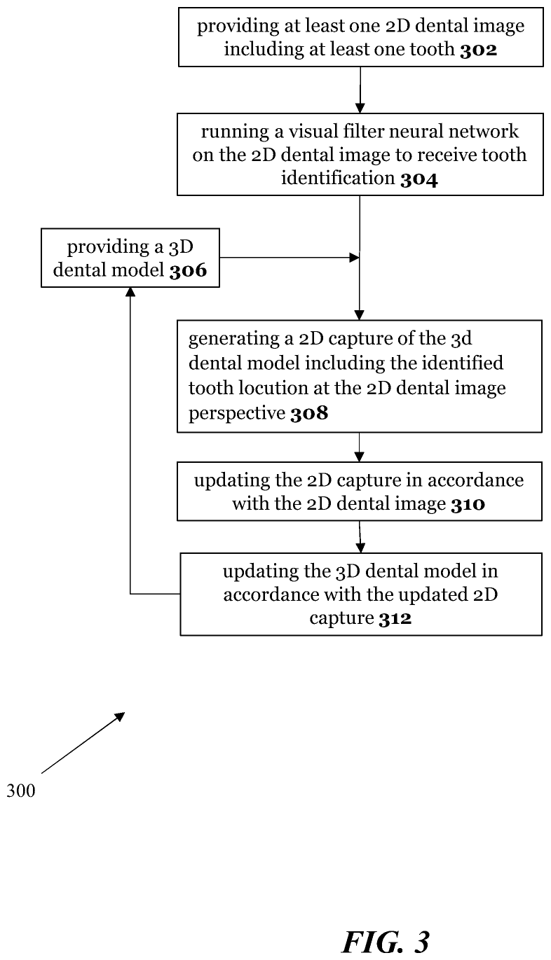

FIG. 3, schematically illustrate an example of a method for updating a three-dimensional (3D) dental model 300 of at least one tooth. In some cases, the method comprises providing at least one 2D dental image including at least one tooth 302. running a visual filter neural network on the 2D dental image to receive tooth identification 304, providing a 3D dental model 306 and generating a 2D capture of the 3d dental model including the identified tooth location at the 2D dental image perspective 308, updating 310 the 2D capture in accordance with the 2D dental image; and updating the 3D dental model 306 in accordance with the updated 2D capture 312.

Methods and systems of the present disclosure beneficially provide a convenient and efficient solution for monitoring and evaluating the positions of a patient's teeth during the course of orthodontic treatment using a user mobile device, in the comfort of the patient's home or another convenient location, without requiring the patient to travel to a dental clinic or undergo a time-consuming and uncomfortable full clinical intraoral dental scan.

In an aspect, the present disclosure provides a method for updating a three-dimensional (3D) model of dental structure, the method comprising: providing a 3D model of dental structure. providing a dental video scan; analyzing the dental video scan to identify at least one tooth, video relative distance or time, and updating the 3D model of dental structure with at least part of the dental structure from the dental video scan.

In some embodiments the analyzing of the dental video scan comprises relative distance between the camera and a selected object on at least two perspectives in the dental video scan.

In preferred embodiments the analyzing comprises identification of at least one arch plane and the relative distance comprises the distance from the arch plane.

In some embodiments the analyzing of dental video scan comprises object distance and time duration of at least two perspectives in the dental video scan.

In preferred embodiments the analyzing of dental video scan comprises identification of at least one focus object in a video frame, generating perspective focus plane and the relative distance from the arch plane.

In some embodiments the updating comprises at least one of the following: (i) structure from motion (SfM) and (ii) multi view stereo (MVS) algorithm of at least two perspectives in the dental video, (iii) determine a transformation for at least one element of the dental structure and applying the transformation to update a position of the at least one element and (iv) deforming a surface of a local area of the at least one element using a deformation algorithm.

In some embodiments the 3D model of dental structure is a generic model

In some embodiments the 3D model is a user dental structure

In some embodiments the relative distance is retrieved from the dental video scan metadata.

In another aspect, the present disclosure provides a non-transitory computer-readable medium comprising machine-executable instructions that, upon execution by one or more computer processors, implements a method for delivering context based information to a mobile device in real time, the method comprising: a memory for storing a set of instructions; and one or more processors configured to execute the set of instructions to: receive a 3D model of dental structure. receive a dental video scan; Analyze the dental video scan to identify at least one tooth, video relative distance or time. Updating the 3D model of dental structure with at least part of the dental structure from the dental video scan.

In some embodiments the analyze of the dental video comprises relative distance between the camera and a selected object on at least two perspectives in the dental video scan.

In some embodiments the analyze comprises wherein the analyzing comprises identification of at least one arch plane and the relative distance comprises the distance from the arch plane.

In some embodiments the analyze of dental video scan comprises object distance and time duration of at least two perspectives in the dental video scan.

In some embodiments the analyze of dental video scan comprises identification of at least one focus object in a video frame, generating perspective focus plane and the relative distance from the arch plane.

In some embodiments the updating comprises at least one of the following: (i) structure from motion (SfM) and (ii) multi view stereo (MVS) algorithm of at least two perspectives in the dental video, (iii) determine a transformation for at least one element of the dental structure and applying the transformation to update a position of the at least one element and (iv) deforming a surface of a local area of the at least one element using a deformation algorithm.

In some embodiments the 3D model of dental structure is a generic model

In some embodiments the 3D model is a user's dental structure

In some embodiments the relative distance is retrieved from the dental video scan metadata.

In an another aspect, provided herein is a method for updating a three-dimensional (3D) dental model of at least one tooth, comprising: (a) providing at least one two-dimensional (2D) dental image including the at least one tooth; (b) running a trained visual filter neural network on the 2D dental image to identify the tooth number of the at least one tooth; (c) providing a baseline 3D dental model that includes the at least one tooth; (d) generating a 2D capture of the baseline 3D dental model; (e) updating the 2D capture of the 3D dental model to include the identified tooth number obtained from the 2D dental image; and (f) using the updated 2D capture to update the 3D dental to include the identified tooth number obtained from the 2D dental image.

In an another aspect, provided herein is a method for updating an initial three-dimensional (3D) dental model of a dental structure of a subject, the method comprising: (a) providing a dental video scan of the dental structure of the subject captured using a camera of a mobile device, wherein the dental structure of the subject comprises one or more oral landmarks; (b) analyzing the dental video scan to identify an oral landmark of the one or more oral landmarks; (c) providing the 3D dental model of the dental structure of the subject; (d) comparing the dental scan video with the 3D dental model to determine differences between the identified oral landmark in the two models; and (e) updating the 3D dental model to include the differences of the identified oral landmark.

In some cases, the analyzing of the dental video scan comprises running a visual filter neural network to identify the tooth number of at least one tooth in the dental structure of the subject. In some cases, the analyzing of the dental video scan comprises determining the relative distance between a camera used to capture the dental video scan and the oral landmark identified in the dental video scan.

In some cases, the identified oral landmark is the arch plane of a subject, and the relative distance comprises the distance from the arch plane to the camera used to capture the dental video scan. In some cases, the analyzing of dental video scan comprises determining the object distance and time duration of at least two perspectives within the dental video scan. In some cases, the analyzing of the dental video scan comprises identifying at least one focus object in a frame of the dental video scan, generating a perspective focus plane of the at least one focus object, and identifying the relative distance from the focus plane to the camera used to capture the dental video scan.

In some cases, the updating comprises: (i) applying structure from motion (SfM) to the dental video scan; (ii) applying a multi view stereo (MVS) algorithm of at least two perspectives to the dental video scan, (iii) determining a transformation of at least one element of the dental structure and applying the transformation to update a position of the at least one element in the 3D dental model; or (iv) deforming a surface of a local area of the at least one element of the dental structure using a deformation algorithm. In some cases, the 3D dental model is a generic model. In some cases, the 3D dental model comprises the dental structure of the subject. In some cases, the relative distance is retrieved from the dental video scan metadata. The present disclosure provides methods and systems that are capable of generating (or configured to generate) a three-dimensional (3D) model of a dental structure of a dental patient using dental scan videos collected using a mobile device. The 3D model may be a 3D surface model (mesh) with fine details of the surface of the dental structure.

In some cases, artificial intelligence, including machine learning algorithms, may be employed to train a predictive model for 3D model, and various other functionalities as described elsewhere herein. A machine learning algorithm may be a neural network, for example. Examples of neural networks that may be used with embodiments herein may include a deep neural network (DNN), convolutional neural network (CNN), and recurrent neural network (RNN).

In some cases, the model may be trained using supervised learning. In some cases, a machine learning algorithm trained model may be pre-trained and implemented on the physical dental imaging system, and the pre-trained model may undergo continual re-training that may involve continual tuning of the predictive model or a component of the predictive model (e.g., classifier) to adapt to changes in the implementation environment over time (e.g., changes in the image data, model performance, expert input, etc.). Alternatively or additionally, the predictive model may be trained using unsupervised learning or semi-supervised learning.

The 3D model generated from the dental scan videos may preserve the fine surface details obtained from the high-resolution clinical intraoral scan while providing accurate and precise measurements of the current position and orientation of a particular dental structure (e.g., one or more teeth). The clinical high-resolution intraoral scanner can use any suitable intra-oral imaging equipment such as a laser or structured light projection scanner.

### 3D Model Generation Algorithm

In an aspect, the present disclosure provides methods for 3D model of a dental structure. At a first point in time, an initial three-dimensional (3D) model general or representing a patient's dental structure is provided by a high-quality intraoral scan as described above. In some cases, the initial 3D model may include a 3D surface model with fine surface details. The initial 3D surface model can be obtained using any suitable intraoral scanning device. In some cases, raw point cloud data provided by the scanner may be processed to generate 3D surfaces of the dental structure (e.g., teeth along with the surrounding gingiva).

At a later point in time during the course of treatment, dental scan videos representing the dental structure may be conveniently provided using a user mobile device. The dental scan videos may be processed to reconstruct a reduced three-dimensional (3D) model of the dental structure. The 3D model may be a dense 3D point cloud that contains reduced 3D information of the dental structure without fine surface details. A transformation between the reduced three-dimensional (3D) model reconstructed from the dental scan video and the initial 3D model (mesh model) is determined by aligning or registering elements of the initial 3D model with corresponding elements within the dental scan video. A three-dimensional (3D) image of the dental structure is subsequently derived or reconstructed by transforming the initial 3D model using the transformation data. The term “rough 3D model” as utilized herein may generally refer to a 3D model with reduced surface details.

In some cases, the data collected from the dental scan video may include perspectives of the dentition (e.g., teeth) from multiple viewing angles. The data may be processed using any suitable computer vision technique to reconstruct a 3D point cloud of the dental structure. The algorithm may include a pipeline for structure from motion (SfM) and multi view stereo (MVS) processing. The first 3D point cloud may be reconstructed by applying structure from motion (SfM) and multi view stereo (MVS) algorithms to the image data. For example, a SfM algorithm is applied to the collected image data to generate estimated camera parameters for each image (and a sparse point cloud describing the scene). Structure from motion (SfM) enables accurate and successful regeneration in cases where multiple scene elements (e.g., arches) do not move independently of each other throughout the image frames. When these scene elements' movements are substantially independent of each other, segmentation masks may be utilized to track the respective movement. The estimated camera parameters may include both intrinsic parameters such as focal length, focus distance, distance between the micro lens array and image sensor, pixel size, and extrinsic parameters of the camera such as information about the transformations from 3D world coordinates to the 3D camera coordinates. Next, the image data and the camera parameters are processed by the multi-view stereo method to output a dense point cloud of the scene (e.g., a dental structure of a patient). In some cases, the dental scan video may be segmented such that each point may be annotated with semantic segmentation information.

The 3D model can be stored in any suitable file formats such as a Standard Triangle Language (STL) file, a WRL file, a 3MF file, an OBJ, a FBX file, a 3DS file, an IGES file, or a STEP file and various others.

In some cases, pre-processing of the dental scan video may be performed to improve the accuracy and quality of the rough 3D model. The pre-processing can include any suitable image processing algorithms, such as image smoothing, to mitigate the effect of sensor noise, image histogram equalization to enhance the pixel intensity values, or video stabilization methods. In some cases, an arch mask may be utilized to track the motion of the arch throughout the video to filter out non-interest anatomical features (e.g., lip, tongue, soft tissue, etc.) in the scene. This beneficially ensures that the rough 3D model (e.g., 3D point cloud) substantially corresponds to the surface of the initial 3D model (e.g., teeth and gum).

In some cases, the pre-processing may be performed using machine learning techniques. For example, pixel segmentation can be used to isolate the upper and lower arches and/or mask out the undesired anatomical features. Pixel segmentation may be performed using a deep learning trained model. In another example, image processing such as smoothing, sharpening, stylization may also be performed using a machine learning trained model. The machine learning network can include various types of neural networks including a deep neural network, convolutional neural network (CNN), and recurrent neural network (RNN). The machine learning algorithm may comprise one or more of the following: a support vector machine (SVM), a naïve Bayes classification, a linear regression, a quantile regression, a logistic regression, a random forest, a neural network, CNN, RNN, a gradient-boosted classifier or repressor, or another supervised or unsupervised machine learning algorithm (e.g., generative adversarial network (GAN), Cycle-GAN, etc.).

The rough 3D model can be reconstructed using various other methods. For instance, the rough 3D model may be reconstructed from a depth map. In some cases, the imaging device may comprise a camera, a video camera, a three-dimensional (3D) depth camera, a stereo camera, a depth camera, a Red Green Blue Depth (RGB-D) camera, a time-of-flight (TOF) camera, an infrared camera, a charge coupled device (CCD) image sensor, or a complementary metal oxide semiconductor (CMOS) image sensor.

In some cases, the rough 3D model regeneration method may include generating the three-dimensional model using one or more aspects of passive triangulation. Passive triangulation may involve using stereo-vision methods to generate a three-dimensional model based on a plurality of images obtained using a stereoscopic camera comprising two or more lenses. In other cases, the 3D model generation method may include generating the three-dimensional model using one or more aspects of active triangulation. Active triangulation may involve using a light source (e.g., a laser source) to project a plurality of optical features (e.g., a laser stripe, one or more laser dots, a laser grid, or a laser pattern) onto one or more intraoral regions of a subject's mouth. Active triangulation may involve computing and/or generating a three-dimensional representation of the one or more intraoral regions of the subject's mouth based on a relative position or a relative orientation of each of the projected optical features in relation to one another. Active triangulation may involve computing and/or generating a three-dimensional representation of the one or more intraoral regions of the subject's mouth based on a relative position or a relative orientation of the projected optical features in relation to the light source or a camera of the mobile device.

In another example, a deep learning model may be utilized to process the input raw image data and output a 3D mesh model. For instance, the deep learning model may include a pose estimation algorithm that can reconstruct a 3D surface model using a single image. Alternatively, the 3D surface model may be reconstructed from multiple images. The pose estimation algorithm can be any type of machine learning network such as a neural network.

### Remote Dental Imaging Platform

As used herein, remote monitoring and dental imaging may refer to monitoring a dental anatomy or a dental condition of a patient and taking images of the dental anatomy at one or more locations remote from the patient or dentist. For example, a dentist or a medical specialist may monitor the dental anatomy or dental condition in a first location that is different than a second location where the patient is located. The first location and the second location may be separated by a distance spanning at least 1 meter, 1 kilometer, 10 kilometers, 100 kilometers, 1000 kilometers, or more. The remote monitoring may be performed by assessing a dental anatomy or a dental condition of the subject using one or more intraoral images captured by the subject when the patient is located remotely from the dentist or a dental office. In some cases, the remote monitoring may be performed in real-time such that a dentist is able to assess the dental anatomy or the dental condition when a subject uses a mobile device to acquire one or more intraoral images of one or more intraoral regions in the patient's mouth. The remote monitoring and dental imaging may be performed using equipment, hardware, and/or software that is not physically located at a dental office.

### Computer Systems

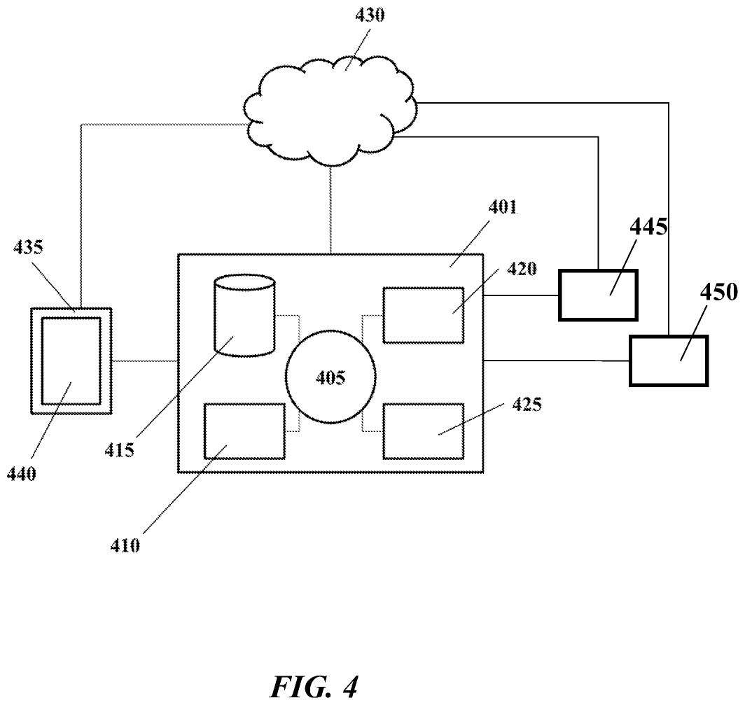

In an aspect, the present disclosure provides computer systems that are programmed or otherwise configured to implement methods of the disclosure. FIG. 4 shows a computer system 401 that is programmed or otherwise configured to implement a method for dental scan, to implement a method for training a neural network, to implement method for designate teeth number or method for updating 3D dental model. The method and implantation can be done in one computer, in few computer systems in different location or in a computer cloud system. The computer system 401 may be configured to, for example, process intraoral videos or images captured using the camera of the mobile device, and designate teeth number to a tooth on dental images. The computer system 401 may be configured to, for example, process a for training a neural network. The computer system 401 may be configured to updating 3D dental model. The computer system 401 can be an electronic device of a user or a computer system that is remotely located with respect to the electronic device. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk, Solid State drive or equivalent storage unit), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental user, or a dentist). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software devices includes optical, electrical and electromagnetic waves, such as used across physical interfaces between local devices, through wired and optical landline networks and over various air-links. The physical devices that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a dental user to view one or more intraoral images or videos captured using a mobile device of the subject or the dental user. The portal may be provided through an application programming interface (API). A user or entity can also interact with various devices in the portal via the UI. Examples of UI's include, without limitation, a graphical user interface (GUI) and web-based user interface.

The computer system 401 can include or be in communication with a Camera 445 for providing, for example, ability to capture videos or images of the subject or a dental user.

The computer system 401 can include or be in communication with a sensor or Sensors 450 including, but not limited to orientation sensor or motion sensor for providing, for example, orientation sensor data or motion sensor data during the dental scan. And for example, retrieve at least one dental scan date (such as acceleration) that can be used to analyzed and compered to at least one dental scan properties

Methods and systems of the present disclosure can be implemented by way of one or more algorithms. An algorithm can be implemented by way of software upon execution by the central processing unit 405.

While embodiments have been shown and described herein, it will be obvious to those skilled in the art that such embodiments are provided by way of example only. It is not intended that the systems and methods described herein be limited by the specific examples provided within the specification. While the systems and methods described herein has been described with reference to the aforementioned specification, the descriptions and illustrations of the embodiments herein are not meant to be construed in a limiting sense.

Numerous variations, changes, and substitutions will now occur to those skilled in the art without departing from the systems and methods described herein. Furthermore, it shall be understood that all aspects of the systems and methods described herein are not limited to the specific depictions, configurations or relative proportions set forth herein which depend upon a variety of conditions and variables. It should be understood that various alternatives to the embodiments described herein may be employed in practicing the systems and methods described herein. It is therefore contemplated that the systems and methods described herein shall also cover any such alternatives, modifications, variations or equivalents. It is intended that the following claims define the scope of the systems and methods described herein and that methods and structures within the scope of these claims and their equivalents be covered thereby.

1. A method for training a visual filter neural network to identify one or more tooth numbers of one or more teeth from one or more dental images, comprising:
(a) providing an intraoral region model, wherein the intraoral region model comprises one or more model teeth;
(b) providing orientation data, wherein the orientation data correlates a spatial location of the one or more model teeth with the corresponding tooth number of the one or more model teeth;
(c) providing a plurality of training dental images, wherein each training dental image of the plurality of training dental images comprises one or more teeth;
(d) creating a plurality of training datasets by using the visual information corresponding to the one or more model teeth to label the one or more teeth in each one of the plurality of training dental images with a respective label, wherein the respective label indicates either a tooth number or a tooth number is not identifiable; and
(e) training the visual filter neural network based on the plurality of training datasets to identify a tooth within a dental image of a subject and label the tooth with a corresponding tooth number.

2. The method of claim 1, wherein the intraoral region model is a two-dimensional (2D) model representation of the intraoral region of a subject from a front perspective or a top view perspective.

3. The method of claim 1, wherein the intraoral region model is a three-dimensional (3D) model representation of the intraoral region of a subject.

4. The method of claim 1, wherein the orientation data is acquired from capturing the intraoral region model with a dental scope, and wherein the orientation data corresponds to the spatial orientation of the dental scope relative to the intraoral region being captured.

5. The method of claim 1, wherein the dental image is captured within the visible light spectrum.

6. The method of claim 1, wherein the dental image is acquired using a dental scope.

7. The method of claim 1, wherein the creating of the plurality of training datasets comprises comparing and matching a rotation or orientation of a tooth in a training dental image with a rotation or orientation of the corresponding model tooth.

8. The method of claim 1, wherein the creating of the plurality of training datasets comprises comparing and matching a scale of a tooth in a training dental image with a scale of the corresponding model tooth.

9. The method of claim 1, wherein the creating of the plurality of training datasets comprises comparing and matching a contour of a tooth in a training dental image with a contour of the corresponding model tooth, wherein a contour of the tooth is determined from outlier pixel intensity values.

10. The method of claim 1, wherein the creating of the plurality of training datasets comprises comparing and matching a color of a tooth in a training dental image with a color of the corresponding model tooth, wherein a color of the tooth is determined from pixel intensity values.

11. The method of claim 1, wherein the creating of the plurality of training datasets comprises comparing and matching morphologic structure of a tooth in a training dental image with a morphologic structure of the corresponding model tooth, wherein the morphologic structure of the tooth is determined from the shape of the teeth and surface pixel color and intensity.

12. The method of claim 1, wherein the creating of the plurality of training datasets comprises identifying a first tooth in the training dental image based on the relation of the first tooth to a second tooth adjacent to or opposite of the first tooth.

13. A method to identify a number of a tooth from a dental image, comprising: providing a dental image, wherein the dental image comprises a visible part of the tooth; and running a visual filter neural network to identify the tooth number.

14. The method of claim 13, wherein the visual filter neural network is provided with an intraoral region model of a user, and wherein the dental image is of the user.

15. The method of claim 14, wherein the dental image is projected on the identified tooth on the intraoral region model of the user.

16. A method for updating a three-dimensional (3D) dental model of at least one tooth, comprising:
(a) providing at least one two-dimensional (2D) dental image including the at least one tooth;
(b) running a visual filter neural network on the 2D dental image to identify the tooth number of the at least one tooth;
(c) providing a baseline 3D dental model that includes the at least one identified tooth;
(d) generating a 2D capture of the baseline 3D dental model;
(e) updating the 2D capture of the 3D dental model in accordance with the 2D dental image; and
(f) using the updated 2D capture to update the 3D dental model.

17. The method of claim 16, wherein the updating comprises: (i) applying structure from motion (SfM) to the dental video scan; (ii) applying a multi view stereo (MVS) algorithm of at least two perspectives to the dental video scan, (iii) determining a transformation of at least one element of the dental structure and applying the transformation to update a position of the at least one element in the 3D dental model; or (iv) deforming a surface of a local area of the at least one element of the dental structure using a deformation algorithm.

18. A non-transitory computer-readable medium comprising machine-executable instructions that, upon execution by one or more computer processors, implements a method for delivering context based information to a mobile device in real time, the method comprising:
a memory for storing a set of instructions; and one or more processors configured to execute the set of instructions to:
(a) provide a dental video scan of the dental structure of the subject using a camera of a mobile device, wherein the dental structure of the subject comprises one or more oral landmarks;
(b) analyze the dental video scan to identify an oral landmark of the one or more oral landmarks;
(c) provide the 3D dental model of the dental structure of the subject;
(d) compare the dental scan video with the 3D dental model to determine differences between the identified oral landmark in the two models; and
(e) update the 3D dental model to include the differences of the identified oral landmark.

19. The method of claim 18, wherein the analyzing of the dental video scan comprises running a visual filter neural network to identify the tooth number of at least one tooth in the dental structure of the subject.

20. The method of claim 18, wherein the analyzing of the dental video scan comprises identifying at least one focus object in a frame of the dental video scan, generating a perspective focus plane of the at least one focus object, and identifying the relative distance from the focus plane to the camera used to capture the dental video scan.
